(19) United States
(12) Patent Application Publication
(10) Pub. No.: US 2003/0104085 A1
(43) Pub. Date: Jun. 5, 2003

(54) Methods and compositions for treating back pain

(76) Inventor: David C. Yeomans, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/006,858

(22) Filed: Dec. 5, 2001

(51) Int. Cl. A61K 31/24; A61K 35/78; A61K 31/16

(52) U.S. Cl. 424/760; 514/537; 514/625

(57) ABSTRACT

The present invention provides methods and kits for treating back pain, especially chronic back pain. The methods comprise administering a vanilloid receptor agonist, preferably a capsaicin.

### BACKGROUND OF THE INVENTION

Current analgesic therapies often fall short of therapeutic goals and typically have unacceptable side effects. In many chronic pain syndromes, such as those subsequent to neuropathic injury, pain is not well controlled by any currently available method. The sensation of pain is transduced in the periphery by pain-sensing, i.e. nociceptive, C- and A-delta primary afferent neurons. These neurons have a peripheral nerve ending in the skin or deep tissues and a central terminal that makes synaptic contact with second order neurons in the spinal cord dorsal horn. The impulse is processed locally for activation of withdrawal reflexes and relayed to the brain for conscious perception and contextually relevant integrated responses.

Vanilloid receptor-1 (VR1) is a multimeric cation channel prominently expressed in nociceptive primary afferent neurons (see, e.g., Caterina et al., Nature 389:8160824, 1997; Tominaga et al., Neuron 531-543, 1998). Activation of the receptor typically occurs at the nerve endings via application of painful heat (VR1 transduces heat pain) or during inflammation or exposure to vanilloids. Activation of VR1 by an agonist, such as resiniferatoxin or capsaicin, results in the opening of calcium channels and the transduction of pain sensation (see, e.g., Szalllasi et al., Mol. Pharmacol. 56:581-587, 1999.) After an initial activation of VR1, VR1 agonists desensitize VR1 to subsequent stimuli. This desensitization phenomenon has been exploited in order to produce analgesia to subsequent nociceptive challenge. For example, it has been shown that topical administration of resinferatoxin (RTX), which is a potent vanilloid receptor agonist, at the nerve endings in the skin triggers a long-lasting insensitivity to chemical pain stimulation. Furthermore, it has been shown that both subcutaneous and epidural administration of the RTX produce thermal analgesia when administered to rats, with no restoration of pain sensitivity for over 7 days (see, e.g., Szabo et al., Brain Res. 840:92-98, 1999).

The effects of intrathecal capsaicin on thermal sensitivity in rats have been investigated. The results, however, have been conflicting (Nagy et al., Brain Res. 211:497-502, 1981; Palermo et al., Brain Res. 208:506-510; Yaksh et al., Science 206:481-483, 1979; and Russell et al., Pain 25:109-123, 1986). Russell et al. observed no thermal analgesia, although in three previous studies, at least some degree of thermal analgesia was observed. The conflicting results raised a number of issues such as the possibility of complications in data interpretation resulting from spinal cord damage from cannula implantation, or solvent toxicity problems.

Low back pain exerts an enormous sociological and economic cost upon the nation. Currently treatments for intractable cases are frequently unsuccessful as well as extremely expensive. These generally involve surgical removal of intervertebral disc material, which is thought to be the source of pain in many cases. Other treatments have included inserting a wire into the disc which is then heated to shrink the disc, and injection of enzyme solutions (particularly papain) into the disc to dissolve it. The former is fairly popular, although there are no well controlled studies that demonstrate efficacy, and the later has been abandoned due to allergic responses.

Under normal conditions, nerve fibers sparsely innervate the surface of discs. After disc injury such as by herniation or a tear, nerve fibers grow from the surface in toward the center. There is also an increase in the number of nerve branches that innervate the disc. These nerve branches show distinct signs of being of the type that carry pain messages, and it is thought that they provide the underlying pathology of so-called “discogenic pain.” These endings express the neuropeptide substance P. Substance P is typically identified with pain transducing neurons and neurons that contain substance P typically express VR1 receptors. Palmgren et al., Spine 21(11):1301-1306 (1996); Palmgren et al., Spine 24(20):2075 (1999); Freemont et al., The Lancet 350:178-181 (1997). One characteristic of many pain neurons is that they are sensitive to capsaicin and similar vanilloids. Lower concentrations can cause pain followed by desensitization. Higher concentrations cause pain, followed by “killing back” of the nerve fibers toward their cells of origin in the dorsal root ganglia.

Treatments for other types of pain, particularly of cutaneous origin, have been developed using capsaicin. Some of these, with relatively low concentrations, are available over the counter as creams and would usually be used in conjunction with local anesthetics, as capsaicin can be quite painful.

### SUMMARY OF THE INVENTION

The present invention features methods for treating discogenic pain by injecting a vanilloid agonist or a formulation comprising a vanilloid agonist into the intravertebral space of patients having discogenic pain. The vanilloid agonist functions by killing back the aberrant nerve fibers, eliminating the source of pain. Based on results in other tissues, such an approach should lead to pain relief for many months, if not permanently. In some embodiments, a vanilloid agonist may be administered on multiple occassions.

Specifically, the present invention is applicable to pain that involves activation of vanilloid receptor-bearing neurons. In particular, selective deletion of nociceptive primary afferent neurons by administration of a vanilloid agonist, e.g., capsaicin or resiniferatoxin (RTX) interrupts the signaling pathway and blocks pain sensation and neurogenic inflammation. This selective application can be used for treatment-resistant back pain. Thus, the invention provides a method of treating pain by ablating pain-sensing neurons.

In some embodiments, the vanilloid receptor 1 agonist is administered to a patient suffering from chronic pain. Preferably, the vanilloid receptor agonist is selected from the group consisting of a resiniferatoxin or a capsaicin, such as ovanil. In especially preferred embodiments, the VR1 agonist is capsaicin. In some embodiments, administration comprises direct injection into the intravertebral disc. In preferred embodiments, the amount of vanilloid agonist administered is sufficient to ablate the neurons. In some embodiments this may be from 50 nanograms to 50 micrograms. Often the amount is from about 500 nanograms to about 50 micrograms. In some embodiments, the method further comprises administering a local anesthetic, often lidocaine or bupivacaine.

In another aspect, the invention provides a kit for treating back pain, said kit comprising a compartment containing a vanilloid receptor agonist in an amount sufficient to ablate the neurons and instructional materials describing how to use the kit. Such a kit can also contain a local anesthetic. In particular embodiments, the vanilloid receptor agonist is a capsaicin.

### DETAILED DESCRIPTION

This invention pertains to the surprising discovery that administration of vanilloid receptor agonist such as a resinaferatoxin or a capsaicin directly into the intravertebral space is useful to selectively treat acute and chronic pain. The method is useful for the treatment of chronic pain, particularly including, but not limited, to back pain.

The term “VR1 agonist” as used herein refers to a to a compound that binds to VR1 and stimulates calcium uptake. Typically, VR1 agonists comprise a vanilloid ring that is important for agonist activity.

The term “discogenic pain” refers to pain, either acute or chronic, originating in one or more vertebrae and being characterized by an ingrowth of nerve fibers into the intravertebral space.

The terms “chronic pain” and “acute pain” incorporate their common usages; subjective means such as clinical diagnosis and objective means such as laboratory tests may be used to determine the presence of chronic pain and/or acute pain, and to distinguish between these two distinct categories of pain.

The term “vanilloid receptor 1” or “VR1” refers to a ligand-gated cation channel, distantly related to the TRP (transient release potential) proteins, that can be activated by vanilloids, heat, and protons. A VR1 agonist binds to VR1 and activates the VR1 cation channel.

The term “pharmaceutically acceptable excipient” incorporates the common usage and refers to includes any suitable pharmaceutical excipient, including, e.g., water, saline, phosphate buffered saline, Hank's solution, Ringer's solution, dextrose/saline, glucose, lactose, or sucrose solutions, magnesium stearate, sodium stearate, glycerol monostearate, glycerol, propylene glycol, ethanol, and the like.

The term “intravertebral administration” refers to administration of a compositions directly into the intravertebral space. In turn “intravertebral space” encompasses the intravertebral disc.

The term “treating” refers to any indicia of success in the treatment or amelioration of an injury, pathology, condition, or symptom (e.g., pain), including any objective or subjective parameter such as abatement; remission; or diminishing of symptoms. For example, the methods of the invention selectively treat chronic pain by ameliorating the hyperalgesia associated with chronic pain, while not significantly affecting non-pain sensory functions.

Diagnosing and Assessing Chronic Pain

The invention provides methods of treating chronic discogenic pain while at the same time not significantly affecting the ability to respond to acutely painful, and potentially harmful, stimuli. Thus, proper diagnosis of chronic pain is necessary both to practice and to assess the success of the compositions and methods of the invention. Means to diagnosis chronic pain include classical clinical and psychological evaluations, which can be augmented by various laboratory procedures, as described herein. Such means are well described in the medical, scientific and patent literature.

One criteria to diagnose a “chronic” pain is whether the pain persists for a month beyond the usual course of an acute disease or a reasonable time for an injury to heal. This evaluation is made by the clinician on a case by case basis. Acute diseases or injuries can heal in 2, 3, or, at most, 6 weeks, depending on the nature of the condition or injury, the age and health of the patient, and the like. For example, a simple wrist fracture can remain painful for a week to ten days; however, if pain persists longer than this period, a dystropathy could be developing which will be irreversible if not treated. See, e.g., Bonica, et al, (1990) “Management of Pain,” 2nd Ed., Vol. 1, Lea & Feibiger, Phil., Pa.; Wall and Melzack (1994) “Textbook of Pain,” Churchill Livingstone, N.Y. Accordingly, a chronic pain is diagnosed by the practitioner based on clinical and laboratory results, depending on the particular condition or injury, patient, and the like (see also, e.g., Russo (1998) Annu. Rev. Med. 49:123-133).

Another means to identify a “chronic” pain is by diagnosis of a pathologic process (which is usually also chronic) known to produce or be associated with chronic pain. Such conditions are well characterized and include, e.g., chronic pain syndrome (see, e.g., Clifford (1993) Can. Fam. Physician 39:549-559), arthralgia, arthritis (e.g., osteoarthritis and rheumatoid arthritis), causalgia, hyperpathia, neuralgia, neuritis, radiculagia, fibromyalgia (see, e.g., Simms (1998) Am. J. Med. Sci. 315:346-350), orofacial pain and temporomandibular disorders (see, e.g., Binderman (1997) Curr. Opin. Periodontol. 4:144-15), reflex sympathetic dystrophy (see, e.g., Dangel (1998) Paediatr. Anaesth. 8:105-112, chronic back pain, certain cancers, and the like.

Chronic pain is also associated with particular injuries to the nerves. These include, e.g., nerve transection (traumatic or surgical), chronic abnormal pressure on a nerve, chemical (e.g., formalin) destruction of nerve tissue, and the like.

Chronic pain can also be distinguished from acute pain by its non-responsiveness to pharmacologic therapies known to significantly ameliorate or abate acute pain. When pain is initially diagnosed as acute or of unknown etiology, the clinician typically administers one of several analgesics known in the art to be effective for acute pain, such as, e.g., a non-steroid anti-inflammatory drug (NSAID), such as, e.g., aspirin, ibuprofen, propoxyphene, tramadol, acetaminophen and the like (see, e.g., Tramer (1998) Acta Anaesthesiol. Scand. 42:71-79). If there is no significant amelioration of pain, as assessed by the clinician, over an approximately six week period, then a provisional diagnosis of chronic pain can be made. Ultimately, as discussed above, a diagnosis of chronic pain depends upon determination as to whether pain would be expected, given each individual situation.

Other treatments to which chronic pain is also typically incompletely or totally unresponsive include tricyclic antidepressant administration, psychotherapy, or alternative medicines, such as acupuncture, biofeedback, and the like.

Laboratory, radiographic and other types of imaging procedures may also be used to diagnose chronic pain. In particular, positron emission tomography, or PET, now allows the clinician to objectify such otherwise merely subjective symptoms, including chronic pain (see, e.g., Reiss (1998) Fortschr. Med. 116:40-43; Di Piero (1991) Pain 46:9-12).

Vanilloid Receptor Agonists

VR1 agonists as defined herein bind to the VR1 receptor and stimulate calcium activity. VR1 agonists are typically characterized by the presence of a vanilloid moiety that mediates binding and activation of the receptor. Any number of VR1 receptor agonists are useful for practicing the methods of the invention. Compounds that act as VR1 receptor agonists include resiniferatoxin and other resiniferatoxin-like complex polycyclic compounds such as tinyatoxin, capsaicin and other capsaicin analogs such as ovanil, and other compounds that include a vanilloid moiety that mediates binding and activation of VR1. In some instances, such as low pH, compounds that lack a vanilloid moiety, e.g., anandamide and the eicosinoids prostacyclin and PGE₂ can also functionally activate VR1.

Resiniferatoxin

In one embodiment, resiniferatoxin (RTX) is used as the vanilloid receptor agonist. RTX, is unlike the structurally related phorbol esters, acts as an ultrapotent analog of capsaicin, the pungent principle of the red pepper. RTX is a tricyclic diterpene isolated from Eurphorbia resinifera. RTX induces pain, hypothermia, and neurogenic inflammation; the acute responses are followed by desensitization to RTX and by cross-desensitization to capsaicin. A homovanillyl group is an important structural feature of capsaicin and the most prominent feature distinguishing resiniferatoxin from typical phorbol-related compounds. Naturally occurring or native RTX has the following structure:

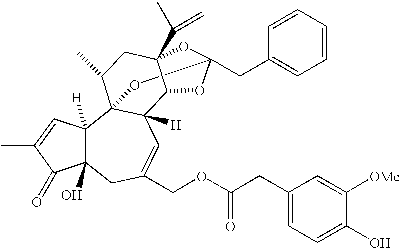

RTX and analog compounds such as tinyatoxin as well other compounds, e.g., 20-homovanillyl esters of diterpenes such as 12-deoxyphorbol 13-phenylacetate 20-homovanillate and mezerein 20-homovanillate, are described, for example, in U.S. Pat. Nos. 4,939,194; 5,021,450; and 5,232,684. Other resiniferatoxin-type phorboid vanilloids have also been identified (see, e.g., Szallasi et al., Brit. J. Phrmacol. 128:428-434, 1999). Often, the C₂₀-homovanillic moiety, the C₃-keto group and the ortho-ester phenyl group on ring C are important structural elements for activity of RTX and its analogs. As used herein, “a resiniferatoxin” or “an RTX” refers to naturally occurring RTX and analogs of RTX, including other phorbol vanilloids with VR1 agonist activity.

Capsaicin

Capsaicin is a natural product in capsicum peppers. As used herein, “a capsaicin” or “capsaicinoids” refers to capsaicin and capsaicin-related or analog compounds. Naturally occurring or native capsaicin has the structure:

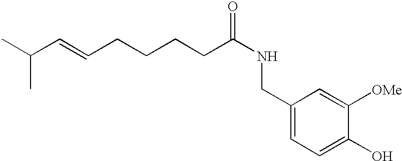

A number of analogs of capsaicins are known in the art including vanillylacylamides, homovanillyl acylamides, carbamate derivatives, sulfonamide derivatives, urea derivatives, aralkylamides and thioamides, aralkyl aralkanamides, phenylacetamides and phenylacetic acid esters are known in the art. In one embodiment, the capsaicin analog olvanil (N-vanillyl-9-octadecenamide) is used in the methods of the invention. Examples of capsaicin and capsaicin analogs are described, for example, in the following patents and patent applications: U.S. Pat. No. 5,962,532; U.S. Pat. No. 5,762,963; U.S. Pat. No. 5,221,692; U.S. Pat. No. 4,313,958; U.S. Pat. No. 4,532,139; U.S. Pat. No. 4,544,668; U.S. Pat. No. 4,564,633; U.S. Pat. No. 4,544,669; and U.S. Pat. Nos. 4,493,848; 4,532,139; 4,564,633; and 4,544,668.

Other VR1 Agonists

Other VR1 agonists such as those described in WO 00/50387) or by Hwang et al., PNAS 97 (11): 6155-6160 (2000) can also be used to selectively ablate C-fiber neurons. Such compounds comprise a vanilloid moiety that mediates binding and activation of VR1. These compounds include compounds having modifications on the C₂₀-homovanillic moiety, the C₃-carbonyl, and the ortho-ester phenyl moiety.

Useful VR1 agonists for practicing the invention can be readily identified using standard methodology. The methodology includes such assessments as measurement of binding to a compound to VR1 and measurement of the ability of the compound to stimulate Ca²⁺ influx. The compound can also be assessed for the ability to kill cells that express the vanilloid receptor. These measurements can be performed using methods known to those of skill in the art.

The ability of a VR1 agonist to bind VR1-bearing cells or membranes can be measured directly or, more typically, in a competition analysis with a known binding compound such as RTX. VR1 binding assays are described in a number of publications, for example WO 00/50387, U.S. Pat. No. 5,232,684, supra; Szallasi et al., Molec. Pharmacol. 56:581-587, 1999. In an exemplary assay, binding activity of a compound containing a vanilloid moiety can be assessed by measuring the ability of the compound to displace bound [³H]RTX from the VR1 receptor. The analysis can be performed using any cell or cell membrane that has VR1 receptors. Often, VR1-expressing transfectants or membranes from the spinal cord are used. The results are usually expressed in terms of Ki values that represent the concentration of the non-radioactive ligand that displaces half of the bound labeled RTX. Preferred VR1 agonists, e.g., RTX, typically have a 10-fold, often a 100-fold, preferably a 1000-fold higher binding affinity for VR1 than native, i.e., the naturally occurring, capsaicin.

In order to identify VR1 agonists, binding assays are typically performed in conjunction with functional assessments that measure the ability of a compound to stimulate changes in membrane potential or changes in calcium influx. Changes in membrane potential or calcium influx can be determined using a variety of assays well known to those in the art. For example, VR1-expressing cells such as neurons from the dorsal root ganglion or VR1 transfectants can be analyzed by patch clamping for changes in whole cell currents that occur upon exposure of the compound being tested for VR1 activity (see, e.g., the Example section below and Caterina et al., Nature 389:816-824, 1997). Another commonly used method to assess VR1 agonist activity is to measure the uptake of calcium using various assays to measure intracellular calcium concentration. For example, calcium flux can be measured by assessment of the uptake of ⁴⁵Ca²⁺ or by using fluorescent dyes such as fura-2. For example, a dye such as fura-2, which undergoes a change in fluorescence upon binding a single Ca²⁺ ion, is loaded into the cytosol of VR1-expressing cells. Upon exposure to VR1 agonist, the increase in cytosolic calcium is reflected by a change in fluorescence of fura-2 that occurs when calcium is bound. Such measurements can also be used to assess the ability of a VR1 agonist to mobilize intracellular calcium stores from the endoplasmic reticulum (ER). In preferred embodiments, VR1 agonists stimulate both a release of Ca²⁺ from the ER and an influx of calcium across the cell membrane.

VR1 agonists of this invention are analyzed for the ability to elicit cell death. In these assays, VR1-expressing cells are exposed to VR1 agonist. VR1-mediated cell death is determined by using morphological assessments and/or staining with vital dyes such as trypan blue (see, e.g., the Examples section and Caterina et al., supra). Preferred VR1 agonists for use in the invention typically are 100 times, often 1000 times more potent than native capsaicin.

Additional compounds e.g., anadamide, and certain eicosanoids such as prostacyclin and PGE2, can activate VR1, but lack a vanilloid moiety. Such compounds can and that are of use in the methods of the invention can also be identified by determining the ability of a compound, to stimulate calcium uptake and/or cause cell death. Such compounds are typically identified in an assay that compares activation of VR1 in response to the compound to activation of VRA in response to a known VR1 agonist, e.g., capsaicin or RTX. comparison to a VR1 agonist that comprises a vanilloid moiety, often in a competitive functional assay. Preferred compounds are 100-fold, preferably 1000-fold, more potent in activating VR1-induced calcium mobilization in comparison to native capsaicin.

Administering VR1 Agonists

VR1 agonists, such as RTX or capsaicin, are formulated as pharmaceuticals to be used in the methods of the invention to treat chronic pain by selective ablation of VR1-expressing neurons. Any VR1 agonist that causes an increase in intracellular calcium, preferably by causing both a transmembrane calcium flux and release of calcium from the ER, and kills VR1-expressing cells can be used as a pharmaceutical in the invention. Routine means to determine VR1 agonist drug regimens and formulations to practice the methods of the invention are well described in the patent and scientific literature, and some illustrative examples are set forth below.

Routes of Administration

The VR1 agonists can be administered by any means that delivers the VR1 agonist into the intravertebral space. These routes of administration include intradiscal, intrathecal and intraganglionic administration (see, e.g., TEXTBOOK OF PAIN, Wall and Melzack, Eds. Harcourt Brace, 4th Ed, 1999). One particularly useful method involves administering by discography as generally described by Carragee et al., Spine 24(23): 2542-2547 (1999).

Determining Dosing Regimens

The pharmaceutical formulations of the invention can be administered in a variety of unit dosage forms, depending upon the particular condition or disease, the degree of chronic pain, the general medical condition of each patient, the method of administration, and the like. In one embodiment, the VR1 agonist is administered in a pharmaceutically acceptable excipient. Details on dosages are well described in the scientific and patent literature, see, e.g., the latest edition of Remington's Pharmaceutical Sciences, Maack Publishing Co, Easton Pa.

The exact concentration of VR1 agonist in a given dose, or the “therapeutically effective dose” may be determined by the medical practitioner. The dosage schedule, i.e., the “dosing regimen,” will depend upon a variety of factors, including the amount of chronic pain present, the duration of the pain, the stage and severity of the disease or condition associated with the chronic pain, and the general state of the patient's health, physical status, age and the like. The state of the art allows the clinician to determine the dosage regimen for each individual patient and, if appropriate, concurrent disease or condition treated. The illustrative example provided below can be used as guidance to determine the dosage regimen, i.e., dose schedule and dosage levels administered when practicing the methods of the invention.

Typically, VR1 agonists are administered to create a temporary environment from about 1 to 5 minutes achieved by injection of the agonist. Based on objective and subjective criteria, as discussed herein, any dosage can be used as required and tolerated by the patient. Multiple administrations can also be performed as required. A typical volume injected is from 50 to 300 microliters delivering a total amount of VR1 agonist that ranges from about 50 nanograms to about 50 micrograms. Often the amount administered is from 200 ng to 1 ug. The VR1 can be administered as a bolus or infused over a period of time, typically from 1 to 5 minutes. The VR1 agonist can be infused over a length of time from about 1 to 5 minutes, or can be delivered as one or more boluses. Dosages in the ranges of 100 nanograms to 500 micrograms are often used. For intrathecal administration, an amount from about 0.5 to 5 ccs, often 3 ccs are injected into the intravertebral space. The total amount of VR1 agonist in the injected volume is usually from about 500 nanograms to about 500 micrograms.

VR1 agonists can be prepared as pharmaceutical compositions by combination with appropriate medical carriers or diluents. Examples of aqueous solutions that can be used in VR1 formulations include water, saline, phosphate buffered saline, Hank's solution, Ringer's solution, dextrose/saline, glucose solutions and the like. The formulations can contain pharmaceutically acceptable auxiliary substances to enhance stability, deliverability or solubility, such as buffering agents, tonicity adjusting agents, wetting agents, detergents and the like. Additives can also include additional active ingredients such as bactericidal agents or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques or can be sterile filtered. The resulting aqueous solutions can be packaged for use as is, or lyophilized, the lyophilized preparation being combined with a sterile aqueous solution prior to administration.

The VR1 agonists are often administered in specific formulations such as isobaric or hyperbaric solutions that may additionally contain other agents such as a long acting local anesthetic. The density of the solution can be controlled using methods known to those of skill in the art. For example, a solution can be made more hyperbaric by the addition of iohexol, iodixanol, metrizamide, sucrose, trehalose, glucose, or other biocompatible molecules with high specific gravity.

In some embodiments, the VR1 agonist is administered in conjunction with a local anesthetic. A local anesthetic refers to a drug that provides temporary numbness and pain relief in a specific region. Local anesthetics are well known to those of skill in the art. These include dibucaine, bupivacaine, ropivacaine, etidocaine, tetracaine, ropivicaine, procaine, chlorocaine, prilocaine, mepivacaine, lidocaine, xylocaine, 2-chloroprocaine, and acid addition salts or mixtures thereof.

The VR1 agonists can also be administered in conjunction with other agents. For example, the VR1 agonist can be administered with a dye or tracer compound when image-guided administration procedures are performed. Common agents include a radio-opaque dye or magnetic resonance contrast agent such as gadlinium.

The VR1 agonists for use to selectively ablate VR1-expressing neurons are administered to a subject such as a mammal, preferably, a primate or a human, but can also be used for other mammals such as horses, cows, sheep, pigs, dogs, cats, rabbits, or other animals.

Kits

After a pharmaceutical comprising a VR1 agonist for use in the methods of the invention has been formulated in an acceptable carrier, it can be placed in an appropriate container and labeled for treatment of an indicated condition, such as chronic back pain. For administration of VR1 agonists, such labeling may include, e.g., instructions concerning the amount, frequency and method of administration. In one embodiment, the invention provides for a kit for the treatment of chronic pain in a human which includes the VR1 agonist and instructional material teaching the indications, dosage and schedule of administration of the agonist. Often, such kits also include a local anesthetic.

It is understood that the examples and embodiments described herein are for illustrative purposes only and that various modifications or changes in light thereof will be suggested to persons skilled in the art and are to be included within the spirit and purview of this application and scope of the appended claims.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill in the art will readily recognize a variety of noncritical parameters that could be changed or modified to yield essentially similar results.

### Example 1

### Presence of Vanilloid Receptors in Human Disc Tissue

Methods: Normal and damaged (typically herniated) vertebral disc tissue is frequently surgically excised in patients with chronic, discogenic pain. After pathologic examination, this tissue is generally discarded. We will obtain normal and damaged disc tissue from clinical laboratories dealing with tissue from these surgeries. This tissue will be sectioned (10 micrometers) and stained for the presence of receptor molecules that respond to capsaicin. Antibodies to these molecules, which are termed VR1 (vanilloid receptor 1) are commercially available and will be used in standard immunohistochemical protocols to demonstrate the presence or absence and the distribution of capsaicin receptors in damaged and normal human disc tissue. Demonstrating VR1 receptor immunoreactivity in disc tissue provides a clear indication that the neurons from which these immunopositive branches arise will be sensitive to capsaicin or other vanilloids. Demonstrating significantly greater density of VR1 immunoreactive branches in damaged tissue indicates a greater sensitivity to capsaicin in these tissues. Specifically, application of capsaicin, at least in some protocols, will selectively kill at least portions of such branches thereby removing a source of discogenic pain.

### Example 2

### Direct Curative Effects of Vanilloid Application to Damaged Disc Tissue

Methods: Rats will be deeply anesthetized with pentobarbital (50 mg/kg, i.p.). A laparotomy will be performed, exposing the ventral surface of the spinal column. After visually identifying particular spinal segments, the tip of a #10 (sharp) scalpel blade will be briefly inserted to a depth of 1.0 mm into the discs of lumbar segments 1-5. It has been clearly shown previously that such injuries produce a dramatic, diffuse overgrowth of branches of pain neurons into cervical discs like the pathology accompanying discogenic pain in human patients. Other (control) rats will have the spinal column exposed, but not injured. The incision wound will be closed in layers with silk suture, and the animals will be allowed to recover in their home cages.

Approximately 4 weeks after injury, the animals will be removed from their cages and placed individually on a rotorod tester. The rotorod allows for motoric testing of rats by placing them on a rotating rod, such that they must walk and coordinate movement to prevent falling. This device serves as an indication of inhibition of movement by pain or hypersensitivity (as happens in human discogenic pain). The duration of time that a rat stays on the rotating rod is used as the index of function. Thus, if the described disc injuries induce movement related pain, the duration of time that the animal remains on the rod significantly decreases. To further exacerbate any changes, the animals can then be ventroflexed (the nose pushed toward the dorsal surface of the hindpaws). This procedure is performed routinely in humans having suspected discogenic pain, and it produces a transient pain that is particularly exacerbated by movement. Thus, if the animals are ventroflexed, there will be a transient further decrease in the time that the animals can remain on the rods.

Some animals will be sacrificed for immunohistochemical analysis of disc tissue at this point (see below). Others will be deeply anesthetized again, and the ventral surface of the spinal column again exposed. Lumbar discs 1-5 will then be injected, through a 28 g needle with either 5.0 μl vehicle, or 0.1 μg capsaicin in 5.0 μl vehicle. These animals will then be resutured in layers and allowed to recover.

One week after administration of capsaicin or vehicle, and weekly for the next 6 weeks, the rats will be retested on the rotorod. It is expected that the application of capsaicin will significantly increase the amount of time that the animals can stay on the rotorod, both with and without retroflexion. At the end of this 6 week period, the rats will be sacrificed so that immunohistochemical analyses can be made of disc tissue.

At the time of sacrifice, the rats will be overdosed with pentobarbital and intracardially perfused with paraformaldehyde fixative. Vertebral discs will be dissected out from the tested lumbar levels from both control and test rats. Discs will be thin-sectioned using a cryostat microtome to allow for immunohistochemical staining. Antibodies specific for small diameter, pain-sensing neurons will be used to detect the location and extent of branching of these neuron filaments in discs. These data will be quantified using a digital image analysis system, to determine whether: 1) the scalpel injury described above induces a significant increase in the number and extent of neuron branches into the disc, relative to sham operated controls; and 2) injection of capsaicin significantly decreases the number and distribution of neuron branches in discs treated this way relative to discs injected with vehicle. These data will then be statistically correlated to the behavioral (rotorod) results to determine whether: 1) scalpel injury to discs both increases neuron branches in the discs and decreases rotorod time; and 2) whether capsaicin, but not vehicle treatment both decreases neuron branches, and increases rotorod time. These results will clearly indicate that capsaicin treatment decreases pain neuron growth into discs and improves motor ability in such a way that is indicative of decreased pain.

What is claimed is:

1. A method of treating pain comprising administering a vanilloid receptor 1 agonist to the intravertebral space in an amount sufficient to ablate neurons.

2. The method of claim 1 wherein the vanilloid receptor 1 agonist is administered to a patient suffering from discogenic pain.

3. The method of claim 1 wherein the vanilloid receptor 1 agonist is selected from the group consisting of a resiniferatoxin or a capsaicin.

4. The method of claim 3 wherein the vanilloid receptor agonist is a capsaicin.

5. The method of claim 1 wherein the amount is from 50 nanograms to 50 micrograms.

6. The method of claim 1 wherein the amount is from about 500 nanograms to about 50 micrograms.

7. The method of claim 1 wherein intravertebral administration comprises direct injection into the space between vertebrae.

8. The method of claim 1 further comprising administering a local anesthetic.

9. The method of claim 8 wherein the local anesthetic is lidocaine, bupivicaine, tetracaine, or ropivicaine.

10. A kit for treating back pain comprising a compartment containing a vanilloid receptor agonist in an amount sufficient to relieve pain and instructional materials describing how to use the kit.

11. The kit of claim 10 wherein the vanilloid receptor agonist is a capsaicin.

12. The kit of claim 10 further comprising a local anesthetic.

13. The kit of claim 12 wherein the local anesthetic is lidocaine or bupivicaine.
